(19) United States
(12) Patent Application Publication
(10) Pub. No.: US 2019/0086362 A1
(43) Pub. Date: Mar. 21, 2019

(54) Electrolyte Solution for Analysis of Biomolecule, Device for Analysis of Biomolecule, and Apparatus for Analysis of Biomolecule

(71) Applicant: Hitachi, Ltd., Chiyoda-ku, Tokyo (JP)

(72) Inventors: Rena AKAHORI, Tokyo (JP); Yusuke GOTO, Tokyo (JP); Kazuma MATSUI, Tokyo (JP); Takahide YOKOI, Tokyo (JP)

(73) Assignee: Hitachi, Ltd., Chiyoda-ku, Tokyo (JP)

(21) Appl. No.: 16/083,147

(22) PCT Filed: Mar. 7, 2017
(86) PCT No.: PCT/JP2017/008897

§ 371 Date: Sep. 7, 2018

(30) Foreign Application Priority Data
Jul. 19, 2016 (JP) 2016-141675

(51) Int. Cl. G01N 27/447; G01N 33/487; C12Q 1/6869

(52) U.S. Cl. CPC G01N 27/44747; G01N 27/44791; C12Q 1/6869; G01N 33/48721

(57) ABSTRACT

A traditional nanopore DNA sequencing method has a problem in that a signal analysis error may occur when a signal variation reflecting fluctuation in a base current is contained in a signal variation in a signal analysis. An electrolyte solution for biomolecule assays, containing D₂O as a solvent, and/or containing an electrolyte that has Cs and Na, Na alone, Na and Li, or Li alone as a cation species in the electrolyte solution, or trishydroxyaminomethane, or a combination thereof is used in formation of a nanopore or in measurement.

### TECHNICAL FIELD

The present invention relates to an electrolyte solution, a device, and an apparatus for use in assays of biomolecules (biopolymers).

### BACKGROUND ART

A technique for electrically measuring a base sequence of a biomolecule (hereinafter referred to as “DNA”) directly without any elongation reaction or fluorescent labeling attracts attentions in the field of next generation DNA sequencers. Specifically, the research and development of nanopore DNA sequencing methods are actively promoted. In the methods, a fragmented DNA chain is directly measured without any reagent to determine the base sequence. Such a method includes directly measuring differences in the base species contained in a DNA chain that is passing through a pore formed in a membrane (hereinafter referred to as “nanopore”) based on the blockage current to sequentially identify the base species. The method neither includes amplification of a template DNA by an enzyme nor uses any labelling substance such as a fluorescent material. Accordingly, such methods are expected as a method that can decode a DNA with a long base sequence at high throughput with a low running cost.

In such nanopore DNA sequencing methods, a variation in the electric resistance caused by an action as carriers of an electrolyte passing through a fine nanopore is acquired by electrodes, and an amplified signal thereof is measured. Hereinafter, the electric current flowing irrespective of the presence or absence of any DNA chain to be measured is called “base current”. The base current basically depends on the size of the nanopore. When a DNA chain is introduced into a nanopore due to electrophoresis, a reduction in the current according to a passing base species is observed. The signal acquired at this time includes a signal component derived from the aforementioned base current and a signal component derived from the size and internal structure of the DNA. In the nanopore DNA sequencing method, a variation in the thus acquired signal is analyzed to identify the sequence of the base species constituting the DNA chain.

### CITATION LIST

### Patent Literature

PTL 1: US patent application publication No. 2009/0286245

### SUMMARY OF INVENTION

### Technical Problem

Meanwhile, in such nanopore DNA sequencing methods, when a signal variation that reflects fluctuation in the base current is contained in a signal variation in a signal analysis, an error may occur in the signal analysis.

### Solution to Problem

For solving the above problem, the present invention adopts a configuration described in claims, for example. The Description includes multiple means for solving the above problem, but one typical example is “an electrolyte solution for biomolecule assays, comprising D₂O as a solvent, and/or an electrolyte that has Cs and Na, Na alone, Na and Li, or Li alone as a cation species in the electrolyte solution, or trishydroxyaminomethane, or a combination thereof”.

### Advantageous Effects of Invention

According to the present invention, abase current measured according to the size of a nanopore formed in a membrane is stabilized, and thus a blockage signal can be stably acquired. Other problems, configurations and advantageous effects than described above will become apparent from the Description of the Embodiments described below.

### BRIEF DESCRIPTION OF DRAWINGS

FIG. 1-1 is a graph showing an image of identification of bases.

FIG. 1-2 shows graphs for explaining a variation in a blockage signal and an analysis result when the base current is stable.

FIG. 1-3 shows graphs for explaining a variation in a blockage signal and an analysis result when the base current is unstable (comparative example).

FIG. 2-1 is a graph showing a measurement example of a base current when H₂O is used as a solvent (comparative example).

FIG. 2-2 is a graph showing a measurement example of a base current when D₂O is used as a solvent.

FIG. 3-1 is a graph showing a measurement example of a base current when a solution according to a comparative example is used.

FIG. 3-2 is a graph showing a measurement example of a base current when a strong alkaline electrolyte solution according to an example is used.

FIG. 4-1 is a graph for explaining composition dependence of a base current when a strong alkaline electrolyte solution is used (comparative example).

FIG. 4-2 is a graph for explaining composition dependence of a base current when a strong alkaline electrolyte solution is used.

FIG. 4-3 is a graph for explaining composition dependence of a base current when a strong alkaline electrolyte solution is used.

FIG. 5-1 is a graph showing a measurement example of a base current when a solution according to a comparative example is used.

FIG. 5-2 is a graph for explaining concentration dependence of abase current when a trishydroxyaminomethane solution is used.

FIG. 5-3 is a graph for explaining concentration dependence of abase current when a trishydroxyaminomethane solution is used.

FIG. 6 is a diagram for explaining a configuration example of a biomolecule assay apparatus of a passive single type.

FIG. 7 is a diagram for explaining a configuration example of a biomolecule assay apparatus of an active single type.

FIG. 8 is a diagram for explaining a configuration example of a biomolecule measurement apparatus of an array passive type.

FIG. 9 is a diagram for explaining a configuration example of a biomolecule measurement apparatus of an array active type.

FIG. 10 is a diagram for explaining a use example of an electrolyte solution.

FIG. 11 is a diagram for explaining a use example of an electrolyte solution.

### DESCRIPTION OF EMBODIMENTS

An embodiment of the present invention will be explained below based on the drawings. Although the appended drawings show specific examples according to the principle of the present invention, the drawings are not used for limiting interpretation of the present invention, but for good understanding of the present invention.

### (1) Summary

As a result of intensive study, the present inventors have found that, when an electrolyte solution that satisfies the following conditions is used as an electrolyte solution for biomolecule assays for use in measurement (hereinafter referred to as “electrolyte solution”), a variation in a base current measured according to the size of a nanopore formed in a membrane can be lowered to 20 pA or less at 50 Hz to 5 kHz, that is, the stability of the base current is increased:
containing D₂O as a solvent, and/or
containing an electrolyte that has Cs and Na, Na alone, Na and Li, or Li alone as a cation species in the electrolyte solution, or trishydroxyaminomethane, or a combination thereof.

The electrolyte solution that satisfies the above conditions can be used as any of (i) a reagent used in acquisition of an ion current via a nanopore opened in a membrane, (ii) a reagent used in opening of a nanopore by application of a voltage to a membrane, (iii) a measurement reagent for assaying a biopolymer constituted of a nucleic acid based on a variation in an electric signal when the biopolymer is passing through a nanopore.

The electrolyte solution preferably satisfies the following (a) to (c):
(a) containing D₂O as a solvent, or
(b) containing an electrolyte that has Cs and Na, Na alone, Na and Li, or Li alone as a cation species in the electrolyte solution, or trishydroxyaminomethane, or a combination thereof, or
(c) a combination of the above (a) and (b).

A device for biomolecule assays used in an assay of a biomolecule constituted of a nucleic acid comprises a first liquid tank and a second liquid tank respectively filled with electrolyte solutions that each satisfy the aforementioned conditions, a membrane separating the first liquid tank and the second liquid tank, and a first electrode and a second electrode respectively provided in the first liquid tank and the second liquid tank. The device for biomolecule assays maybe configured as an array device. An array device refers to a device provided with plural pairs of liquid chambers each separated by a membrane. For example, the first liquid tank is a common tank, and the second liquid tank is a plurality of individual tanks. In this case, an electrode is placed in each of the common tank and the individual tanks. A biomolecule assay apparatus includes a measuring unit for measuring an ion current (blockage signal) flowing between the electrodes provided in the device for biomolecule assays, and acquires sequence information of a biomolecule based on the measured ion current (blockage signal).

The use of the aforementioned solution leads to securement of stability of a base current, realizing stable acquisition of ion currents (blockage signals). As a result, the solution is useful in assays of biomolecules constituted of nucleic acids, and in the fields of tests, diagnoses, treatments, drug productions, and basic studies utilizing the assay information. The presence of a signal variation of 50 pA/0.1 V or less was observed in a base current in a stage before introduction of a biomolecule.

### (2) Electrolyte Solution

As described above, in a method in which a biomolecule is assayed using a so-called blockage current method, as an electrolyte solution in contact with a membrane having a nanopore, a solution satisfying “containing D₂O as a solvent” and/or “containing an electrolyte that has Cs and Na, Na alone, Na and Li, or Li alone as a cation species in the electrolyte solution, or trishydroxyaminomethane, or a combination thereof” is used.

The use of this solution leads to enhancing stability of a base current flowing via a nanopore, and thus a feature of a biomolecule acquired when the biomolecule is passing through the nanopore can be accurately analyzed. As a measure of the stability, for example, a reduction to 20 pA or less at 50 Hz to 5 kHz can be realized. The contribution of the conditions to stabilization of a base current will be specifically explained below.

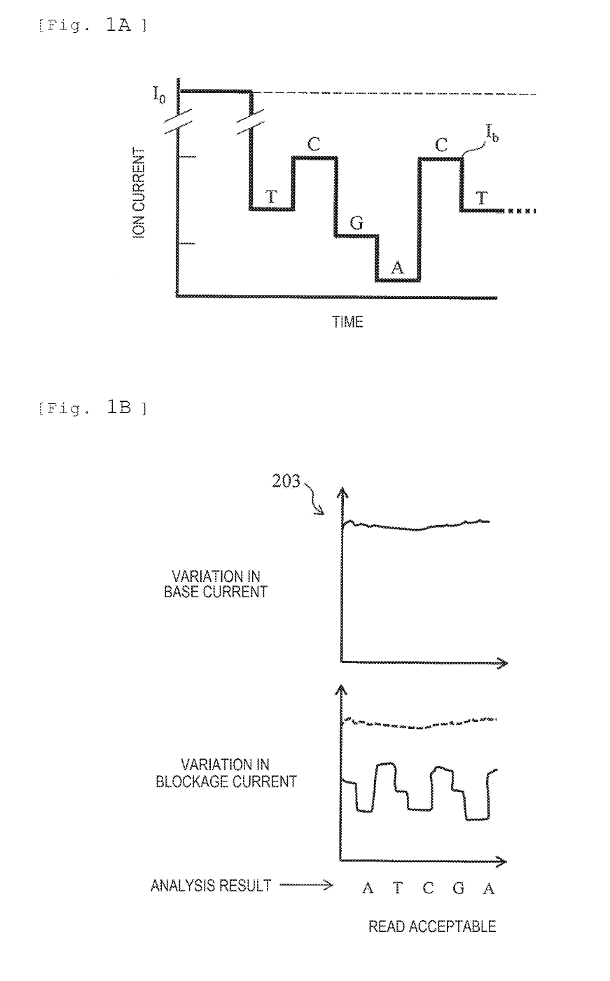

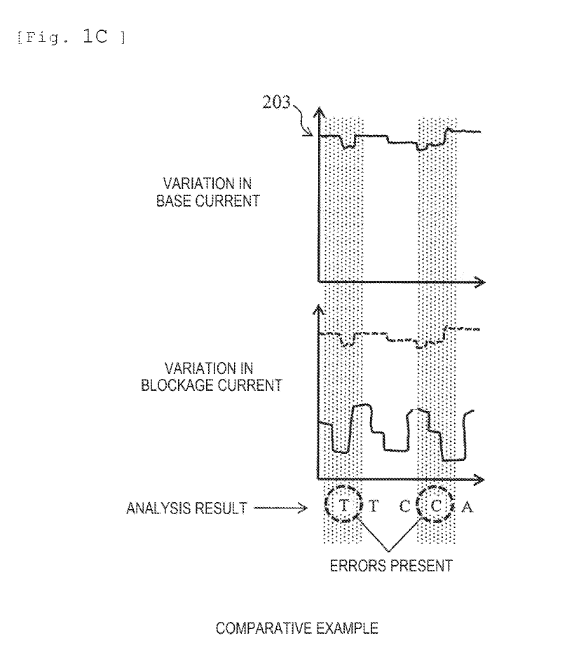

FIG. 1-1 shows an image of identification of bases. A device for biomolecule assays for use in measurement of an ion current by a blockage current method comprises a pair of liquid tanks facing each other with a membrane having a nanopore formed therein in between and a pair of electrodes corresponding to the respective liquid tanks. In measurement, a voltage is applied between the pair of electrodes, and a current flows between the electrodes in the respective liquid tanks. In an initial stage of the application of the voltage, a biomolecule is not introduced in the nanopore, and therefore a current corresponding to the size (pore diameter) of the nanopore is measured. This current corresponds to a base current I₀. Subsequently, a biomolecule contained in the electrolyte solution is introduced into the nanopore due to the difference in potential generated between the two ends of the nanopore.

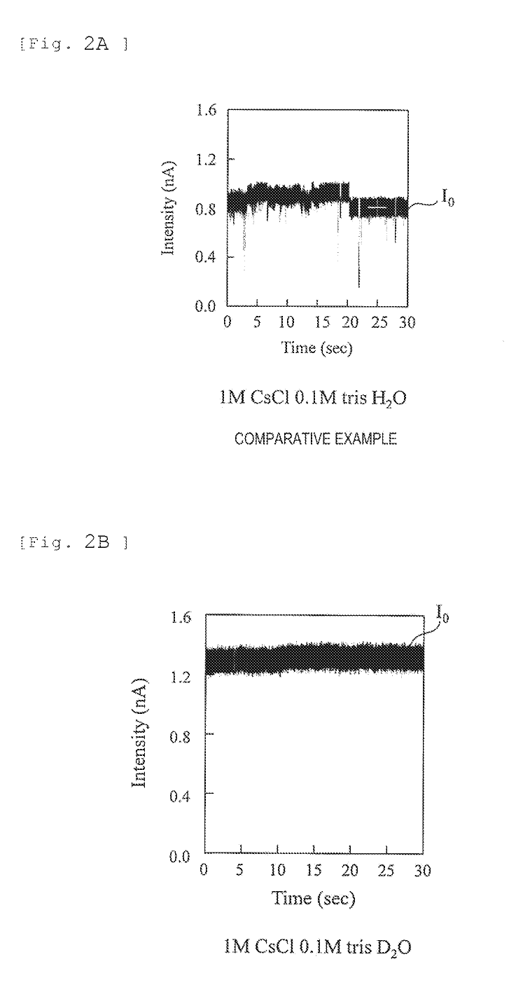

During the biomolecule (particularly polymeric biomolecule) introduced into the nanopore is passing, the biomolecule acts as a resistance to decrease the current flowing through the nanopore. The current flowing at this time corresponds to a blockage current I_{b}. As shown in FIG. 1-1, the blockage current I_{b} is a value smaller than the base current I₀. As shown in FIG. 1-1, the current value of the blockage current I_{b} varies according to the monomer species constituting the polymeric biomolecule. FIG. 1-2 represents a variation in the blockage current I_{b} when the variation in the base current I₀ is small. FIG. 1-3 represents a variation in the blockage current I_{b} when the variation in the base current I₀ is large. As shown in FIG. 1-3, when a noise is present in the base current I₀, a noise of the same amount is also present in the blockage current I_{b}. Since the biomolecule measurement apparatus analyzes the monomer species by identifying a variation in the blockage current I_{b}, a variation in the base current I₀ may cause an error in the analysis. In order to eliminate errors in the analyses, an amplitude of variation ΔI has to be 50 pA or less at 1 kHz or higher.

It is considered that a random telegraph noise (RTN) causes a noise which is contained in the base current I₀. RTN is generally observed in a semiconductor device, and one reason thereof is said to be occurrence of a coupling or separation of an electron with or from a film defect formed in an interlayer insulating film. On the other hand, also in a nanopore formed by application of a voltage, a similar phenomenon is possibly observed through coupling or separation of a proton, a cation, or an anion in an electrolyte solution to or from a defect formed in the membrane after formation of the nanopore. Accordingly, stabilization of the base current I₀ could be expected to be achieved by controlling the ion species that accesses such a membrane defect.

Thus, in this example, an electrolyte solution “containing D₂O as a solvent” and/or “containing an electrolyte that has Cs and Na, Na alone, Na and Li, or Li alone as a cation species in the electrolyte solution, or trishydroxyaminomethane, or a combination thereof” is used as a solution for formation of a nanopore or a measurement solution in measurement.

It will be first confirmed that the use of D₂O as a solvent results in the effect of stabilizing the base current I. FIG. 2-1 is an example of acquisition of the base current I₀ when H₂O is used as a solvent. In this case, a blockage-like signal or fluctuation was observed in the base current I₀ before introduction of a biomolecule. FIG. 2-2 shows an example of acquisition of the base current I₀ when D₂O is used as a solvent. In this case, the same phenomenon as in the case of H₂O was not observed. The amplitude of variation in the base current I₀ satisfies 50 pA or less at 1 kHz or higher.

Stabilization of the base current I₀ by the use of D₂O as a solvent of an electrolyte solution is surmised from the contribution of deuterium to an effect of increasing reliability of a super thin film gate. For example, it is reported that reduction in defects in a gate oxide film or an Si interface is recognized when D₂ pyrogenic oxidation and film formation by deuterium silane gas on a gate electrode polycrystal silicon are utilized in formation of the gate oxide film (Toshiba Review, vol. 57, No. 11 (2002)). This is supposedly because stable deuterium bonds are formed throughout the silicon super thin gate oxide film in the course of the growth of the oxide film, and are adsorbed well to a film defect formed in a semiconductor produced when an electric stress is applied.

A similar phenomenon possibly occurs for a defect formed in a membrane after application of a voltage in the formation of a nanopore. It is supposed that an electrolyte whose solvent is D₂O be adsorbed well to a defect formed in a membrane, which leads to reduction of a phenomenon of adsorption and desorption of a proton in the solution, resulting in stabilization of the base current I₀.

The solvent of the electrolyte solution does not have to be only D₂O, and D₂O may constitute a part of the solvent. As a solvent mixed with D₂O, a solvent that can stably disperse a biopolymer, does not dissolve electrodes, and does not inhibit transfer of electrons with electrodes may be used. Examples include H₂O, alcohols (methanol, ethanol, isopropanol, etc.), acetic acid, acetone, acetonitrile, dimethylformamide (DMF), and dimethylsulfoxide (DMSO). When a nucleic acid is to be measured as a biopolymer, water is the most preferred.

The effect of stabilizing the base current I₀ obtained when the electrolyte dissolved in the electrolyte solution is the aforementioned substance will be explained next. In the case of this example, a cesium, potassium, rubidium, sodium, or lithium cation which is a monovalent cation is used as an electrolyte. At this time, an electrolyte containing a combination of two of the cations also showed an effect of stabilizing the base current I₀.

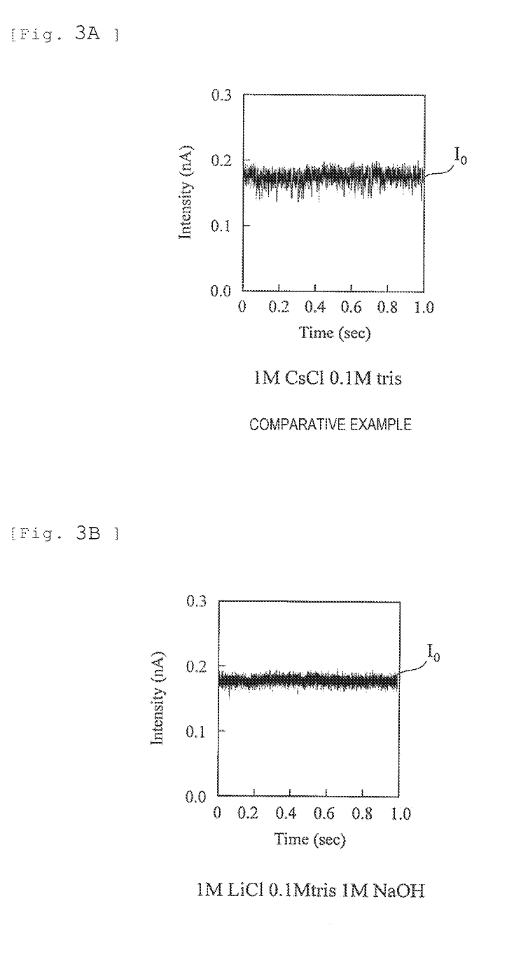

FIG. 3-1 shows an example of acquisition of the base current I₀ when a 1 M CsCl 0.1 M tris solution (pH=10.6) is used. FIG. 3-2 shows an example of acquisition of the base current I₀ when an electrolyte solution obtained by adding 1M NaOH to 1 M LiCl 0.1M tris (pH=11.8) is used. FIG. 3-2 shows an example of a strong alkaline electrolyte solution corresponding to the examples. Both the data were subjected to a software filter of 2 kHz.

The characteristics of the base current I₀ in the case of using a conventional solution (FIG. 3-1) is Ipp=0.1 nA, S.D.=0.026. On the other hand, the characteristics of the base current I₀ in the case of using a strong alkaline electrolyte aqueous solution (FIG. 3-2) is Ipp=0.03 nA, S.D.=0.0074 without any variation of 20 to 50 pA in the baseline.

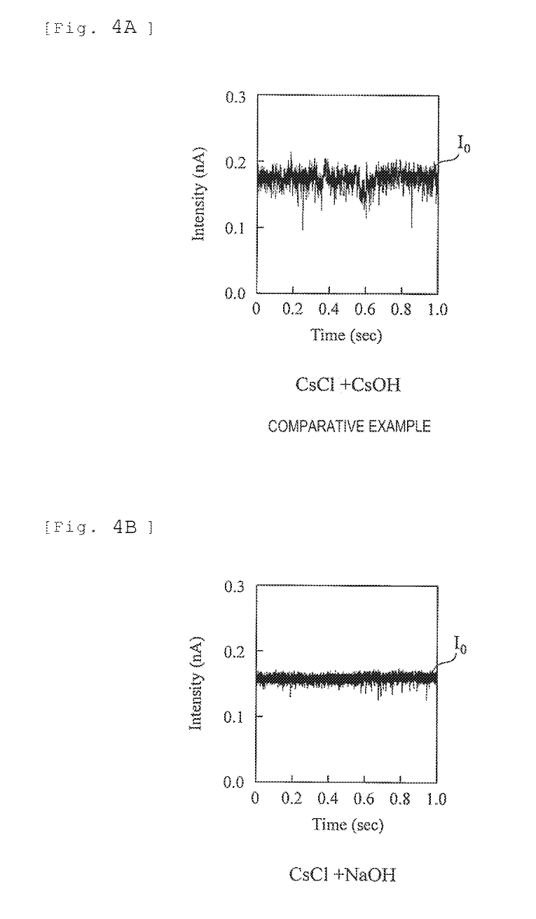

It has been found that the effect on this variation is changed depending on, not only pH, but also the combination of cation species. FIGS. 4-1 to 4-3 show variations in the base current I₀ under various cation species. FIG. 4-1 shows a variation in the base current I₀ when cesium chloride and cesium hydroxide are combined, FIG. 4-2 shows a variation in the base current I₀ when cesium chloride and sodium hydroxide are combined, and FIG. 4-3 shows a variation in the base current I₀ when lithium chloride and sodium hydroxide are combined.

As can be seen in comparison of the drawings, the combination of cesium chloride with sodium hydroxide (FIG. 4-2) or the combination of lithium chloride with sodium hydroxide (FIG. 4-3) show higher stability of the base current I₀ as compared with the combination of cesium chloride with cesium hydroxide (FIG. 4-1). The primary reason why the combinations stabilize the base current I₀ is unclear. The main body to exhibit the effect depends on the ratio of amounts of the cation species used.

The ionic radii of cations decrease in the order of cesium, potassium, rubidium, sodium, lithium. It is confirmed that the tendency of the ionic radii substantially coincides with the tendency of the current stabilization. If the effect of suppressing RTN is attributable to the influence of desorption or adsorption of ions or electrons from or to a defect formed in an SiN membrane, the reason is supposed to be that the adsorption onto a defect increases or significantly decreases as the ionic radius of a cation decreases. Accordingly, cation species having smaller ionic radii are preferably incorporated in a larger proportion in the electrolyte solution.

Some biomolecules have poor resistance to alkali. The contribution to the stabilization of the base current I₀ is however sufficiently maintained even under a neutral condition, for example, in 1 M LiCl.

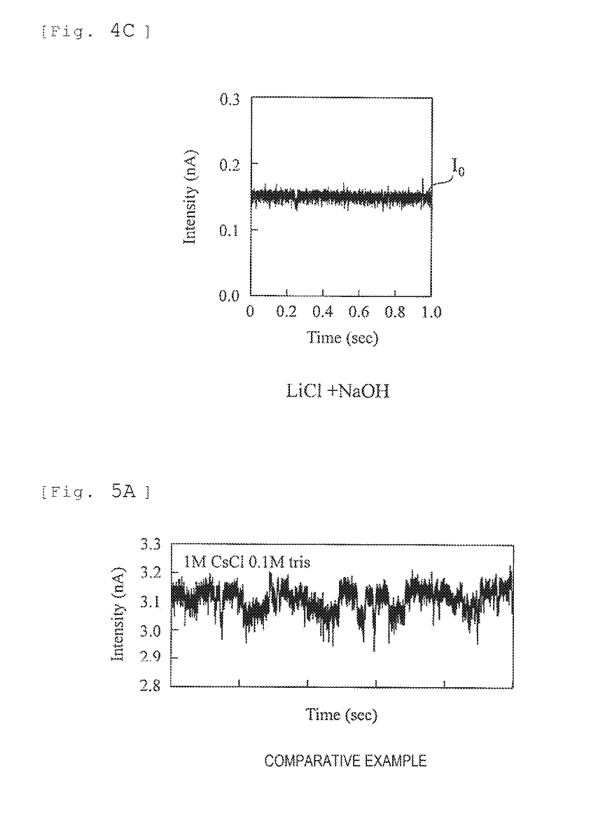

Other combinations of electrolytes that stabilizes the base current I₀ include one containing trishydroxyaminomethane (tris(hydroxymethyl)aminomethane). FIG. 5-1 shows a measurement example of the base current I₀ when a solution according to a comparative example is used. The solution used here is 1 M CsCl 0.1 M tris which is the same as the solution used in the measurement of FIG. 3-1. FIGS. 5-2 and 5-3 are measurement examples of the base current I₀ when a trishydroxyaminomethane solution is used. FIG. 5-2 shows the base current I₀ in a measurement with 1 M CsCl 2 M tris, and FIG. 5-3 shows the base current I₀ in a measurement with 1 M CsCl 1M NaOH. S.D. values in the graphs are 0.04, 0.017, and 0.018, respectively, and the same effect as in the case of the measurement with a strong alkaline electrolyte solution was obtained.

As described above, the electrolyte solution proposed in this embodiment can be used for formation of a nanopore. In a device for biomolecule assays using a membrane having a nanopore formed by using the electrolyte solution, there are a small number of defects involved in the phenomenon of adsorption and desorption of protons, as described above. Accordingly, even when an existing electrolyte solution is used as an electrolyte solution in measurement, the measurement can be performed in the state where the base current I₀ is stable. It is possible that an existing technique is used for formation of a nanopore and the electrolyte solution according to the example is used as the electrolyte solution in measurement. Of course, the electrolyte solution according to the example can be used both for formation of a nanopore and for measurement.

A case where the electrolyte is changed after formation of a nanopore will be explained below. As an alternative cation for metal ions, organic cations constituted of organic materials may be used in this case, and, for example, ionizable cations, such as an ammonium ion, can be used.

As an anion, ionizable anions can be used and are preferably selected based on compatibility with the electrode material. For example, when a silver halide is used as an electrode material, a halide ion (chloride ion, bromide ion, iodide ion) is preferably used as an anion. The anion may be organic anions, such as glutamate ion.

Subsequently, a method for determining the pH of the electrolyte solution will be explained. In the case of this example, the pH of a measurement solution is made to a pKa of a guanine base or higher to pH 14 or lower. Here, the pKa of the guanine base (N-1 position) varies also by the solute species coexisting in the solvent, and thus the pH is preferably adjusted according to the type of the measurement solution. Typically, the pKa of the guanine base N-1 position is 9.2 (for example, Fedor, et al., Nature Reviews Molecular Cell Biology, 6(5): 399-412, 2005).

The upper limit of the pH value is determined in the following manner. The upper limit of the pH value of a measurement solution is determined by the limit of tolerance of the device and the limit of tolerance of the polymeric biomolecule to be measured. The limit of tolerance of the device is around pH 14 where etching of silicon is started when a silicon wafer which is typically used in a semiconductor nanopore is used as a substrate. Such an etching rate is already known (Lloyd D. Clark, et al. Cesium Hydroxide (CsOH): A Useful Etchant for Micromachining Silicon, Technical Digest, Solid-State Sensor and Actuator Workshop, IEEE, 1988).

Silicon nitride which is often adopted as a membrane material is never etched even in a pH in a high alkali region. However, since silicon or silicon oxide as a base material is gradually etched, the upper limit value of the pH is preferably set to 14. In another semiconductor material, the upper limit is determined depending on the limit of tolerance of the device of the material in the same way.

On the other hand, in polymeric biomolecules (particularly DNA and the like), it is known that scission of the long chain is observed when sodium hydroxide (NaOH) is contained at 0.3 M or more in the solution. A hydroxide solution of a different cation species that is known to have concentration dependence provides the same result. When the polymeric biomolecule is DNA, pH 12 or less is desired.

Meanwhile, a measurement solution, if in contact with the atmosphere, causes a phenomenon that the pH gradually transfers to the acidic side due to a reaction with carbon dioxide in the atmosphere. In order to reduce the influence of the carbon dioxide, the pH is set to a higher alkali side from an initial stage, or the concentration of a pH modifier is increased. Specifically, in comparison between 10 mM and 100 mM of a pH modifier, the time until the pH reaches a pKa of a guanine base or lower from the same pH is longer in 100 mM. Thus, a higher concentration of a pH modifier is preferred, and the concentration is preferably 50 mM or more, and more preferably 100 mM or more.

The method for adjusting the pH to the alkali side will be explained. The measurement solution according to the example can be prepared by a known method. For example, the solution can be prepared by dissolving an electrolyte in a solvent, and then adjusting the pH by an appropriate means.

In addition, from the viewpoint of the signal-to-noise ratio, a lower limit is preferably set for the concentration of the electrolyte. In this embodiment, the lower limit of the electrolyte concentration has to be 10 mM. On the other hand, there is no factor to inhibit the upper limit of the electrolyte concentration, and any concentration to the saturation concentration is allowable. That is, the cesium ion concentration in the measurement solution is 10 mM or more and the saturation concentration or less. The concentration is preferably 0.1 M or more and the saturation concentration or less.

It has been found that the electrolyte solution according to this embodiment exhibits the effect of stabilization of the base current if it is used in formation of a nanopore, even in an analysis of the biomolecule using another solution than the aforementioned solution. Thus, the composition of the solution used for formation of a nanopore can be different from that of the solution used in a measurement. A solution used in an analysis of a biomolecule preferably includes such a cation species and has such a pH that do not contribute to formation of three dimensional structure of a molecule to be measured.

The measurement solution for assaying a biopolymer according to this embodiment contains the measurement solution described above as a component. The measurement solution may be provided together with an instruction on which a procedure for use, an amount to be used, or the like are written. The measurement solution may be provided in a ready-to-use state (liquid), may be provided as a concentrated liquid that is to be diluted with a suitable solvent in use, or may be provided in a solid form (for example, powder) that is to be reconfigured with a suitable solvent in use. Such forms and preparations of the measurement solution will be understood by those skilled in the art.

### (3) Device for Biomolecule Assays and Biomolecule Assay Apparatus

Hereinunder, a device for biomolecule assays including a membrane having a nanopore formed using the aforementioned electrolyte solution, or a device for biomolecule assays in which the aforementioned electrolyte solution is used as a measurement solution, and an apparatus for assaying a biomolecule using the device will be explained.

### (3-1) Passive Single Type

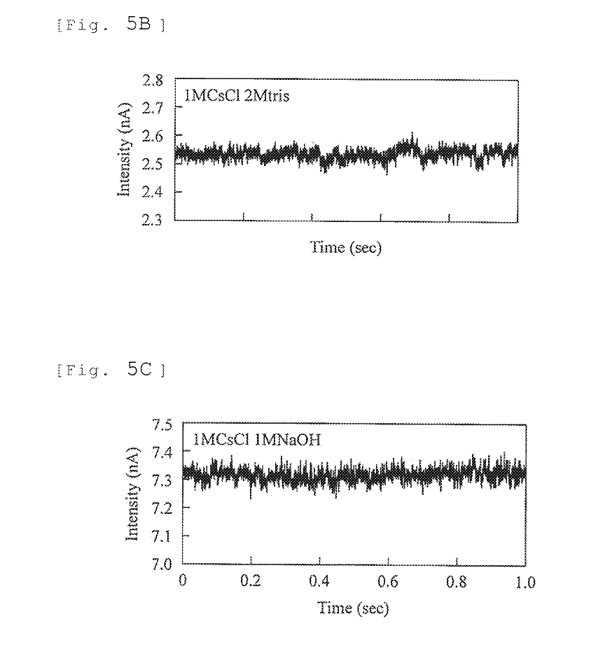

FIG. 6 shows a configuration example of a biomolecule assay apparatus 100 comprising a single-use type device for biomolecule assays 110, a power source 120, an ammeter 121, and a computer 130. The device for biomolecule assays 110 includes two liquid tanks 112A and 112B separated by a partition 111. The partition 111 comprises a membrane 111A having a nanopore 113 formed therein and membrane fixing members 111B and 111C for the membrane 111A. The nanopore 113 may be formed at any position of the membrane 111A. In this embodiment, only one nanopore 113 is provided.

The membrane fixing member 111B and the membrane 111A constitutes a part of the structure of the liquid tank 112A. The membrane 111A and the membrane fixing member 111C constitute a part of the structure of the liquid tank 112B. A through hole is formed in a central portion of the membrane fixing members 111B and 111C, the membrane 111A having the nanopore 113 formed therein is in contact with an electrolyte solution 114 in the part of the through hole.

The membrane 111A exposed in the part of the through hole provided in the membrane fixing members 111B and 111C has to have such an area that two or more nanopores 113 are hardly formed in formation of the nanopore 113 by application of a voltage, and that is allowable in terms of the strength. The area is, for example, approximately 100 to 500 nm². A suitable thickness ranges from such a thickness that provides an effective thickness corresponding to one base and that allows for formation of the nanopore 113 to approximately 7 nm for achieving the DNA one base resolution.

The “single type”, as used herein, refers to an apparatus configuration where one liquid tank 112A and one liquid tank 112B are disposed with the membrane 111A in between as shown in FIG. 6. As used herein, the “passive type” refers to an apparatus configuration that has no movable part in the apparatus, and the “active type” refers to an apparatus configuration that has a movable part in the apparatus. Accordingly, the biomolecule assay apparatus 100 shown in FIG. 6 is classified into a passive single type.

Both of the liquid tank 112A and the liquid tank 112B are filled with the electrolyte solution 114. In this embodiment, the volume of the electrolyte solution 114 is in the order of microliters or milliliters. The liquid tank 112A is provided with an inlet (not shown), and the electrolyte solution 114 which is a DNA solution containing DNA chains 116 can be injected through the inlet. That is, a DNA solution to be measured is injected into the liquid tank 112A positioned on the upper side of the drawing. The same is applied to another type described later.

For the electrolyte solution 114, for example, KCl, NaCl, LiCl, CsCl, or MgCl₂ is used. 4 M or more of Urea, or DMSO, DMF, or NaOH may be mixed in such a solution for suppressing formation of a self-complementary chain of the biomolecule. A buffer can be mixed therein for stabilizing the biomolecule. As a buffer, Tris, EDTA, PBS, or the like is used.

The liquid tank 112A is provided with an electrode 115A and the liquid tank 112B is provided with an electrode 115B. The electrodes 115A and 115B are made of, for example, Ag, AgCl, or Pt, and are in contact with the electrolyte solution 114. Although not shown in FIG. 6, connection terminals which are electrically connected to the electrodes 115A and 115B are provided on the circumference of the device for biomolecule assays 110, and are connected to the power source 120 and the ammeter 121.

When a voltage is applied between the electrode 115A and the electrode 115B, a potential difference is generated between the two surfaces of the membrane 111A having the nanopore 113 formed therein, and the DNA chains 116 dissolved in the liquid tank 112A on the upper side undergoes electrophoresis toward the liquid tank 112B positioned on the lower side. The ammeter 121 includes an amplifier for amplifying a current flowing between the electrodes due to application of a voltage and an analog-to-digital converter (ADC). A detected value which is an output of the ADC is output to the computer 130. The computer 130 collects and records the detected current value. The configuration does not have to be a configuration as shown in FIG. 6 where the power source 120, the ammeter 121, and the computer 130 are separately provided from the device for biomolecule assays 110, and may be an integral configuration where the power source 120, the ammeter 121, and the computer 130 are integrated with the device for biomolecule assays 110.

Here, the device for biomolecule assays 110 is distributed, for example, in the following form. Basically the same is applied to the device for biomolecule assays 110 of another type described later.
(a) The device for biomolecule assays 110 that includes the membrane 111A having the nanopore 113 processed using the electrolyte solution 114 having the composition according to the example explained in “(2) Electrolyte Solution”. In this case, the liquid tanks 112A and 112B may be or may not be filled with an electrolyte solution.
(b) The device for biomolecule assays 110 that includes the liquid tanks 112A and 112B filled with the electrolyte solution 114 having the composition according to the example explained in “(2)Electrolyte Solution”. In this case, any method can be used for formation of the nanopore 113.

### (3-2) Active Single Type

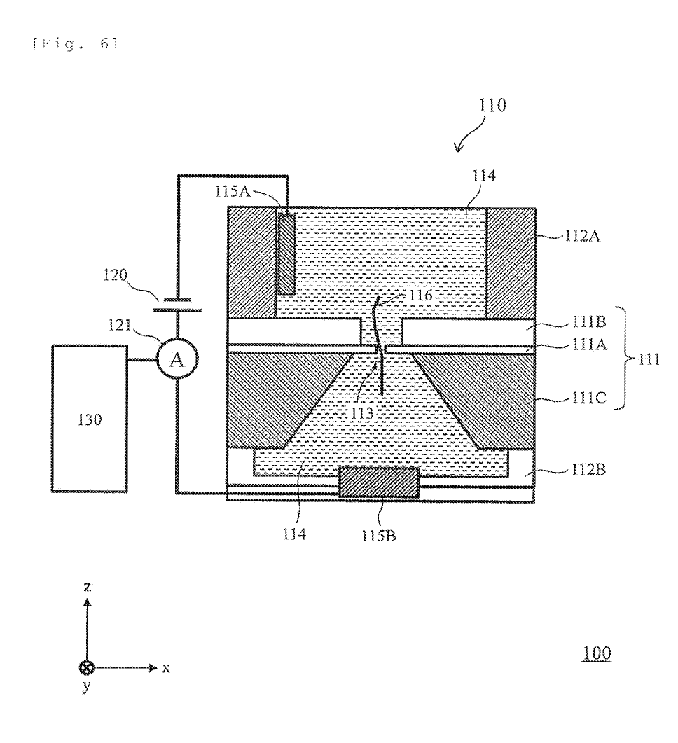

FIG. 7 shows a configuration example of a biomolecule assay apparatus 200 of an active single type. In FIG. 7, the same sign is added to denote a component corresponding to a component in FIG. 6. A basic configuration of the device for biomolecule assays 110A in this example is the same as for the passive single type described above. However, an opening is formed in a part of the outer wall of the liquid tank 112A constituting the device for biomolecule assays 110A, and a driving mechanism 201 is attached to the opening.

A biomolecule immobilizing member 202 is attached on the lower surface side of the driving mechanism 201. The DNA chains 116 are immobilized on the surface facing the membrane 111A of the biomolecule immobilizing member 202. The size of the surface on which the DNA chains 116 are immobilized is larger than the size of a part of the membrane 111A that is in contact with the electrolyte solution 114. The biomolecule immobilizing member 202 is moved upwardly and downwardly in the drawing by an operation driven by the driving mechanism 201. In other words, the surface of the biomolecule immobilizing member 202 on which the DNA chains 116 are immobilized is moved closer to or further from the membrane 111A. In an active type, the DNA chain 116 is introduced into the nanopore 113 by the surface of the biomolecule immobilizing member 202 coming closer to the membrane 111A.

The operation of the driving mechanism 201 is controlled by a control unit 203. The contact of the biomolecule immobilizing member 202 with the membrane 111A is prevented by the membrane fixing member 111B. The membrane 111A may be broken when the biomolecule immobilizing member 202 comes into contact with the membrane 111A having the nanopore 113 formed therein. That is, the membrane fixing member 111B also functions as a means for stopping the lowering of the biomolecule immobilizing member 202. For this reason, the membrane fixing member 111B surrounds the circumference of the membrane 111A like a bank to form a space between the biomolecule immobilizing member 202 and the membrane 111A. A circular through hole is formed in a central portion of the membrane fixing member 111B and the nanopore 113 is disposed inside the through hole.

As described above, the size of the membrane 111A positioned inside the through hole provided at the center of the membrane fixing member 111B is smaller than the size of the lower surface side of the biomolecule immobilizing member 202. For this reason, when the biomolecule immobilizing member 202 is lowered, the lower surface hits the membrane fixing member 111B before coming into contact with the membrane 111A. This stops the lowering of the biomolecule immobilizing member 202, and thus the contact of the biomolecule immobilizing member 202 with the membrane 111A is prevented. That is, breakage of the membrane 111A is avoided. A suitable thickness of the membrane fixing members 111B and 111C is approximately 200 to 500 nm in view of securement of the strength of the membrane 111A and the fluctuation in the immobilization height of the biomolecule immobilized on the surface of the biomolecule immobilizing member 202. In this embodiment, the membrane 111A has a diameter of 500 nm and the membrane fixing members 111B and 111C have a thickness of 250 nm.

The biomolecule immobilizing member 202 can be fixed to the driving mechanism 201 by vacuum adsorption or pressure bonding. The driving mechanism 201 is formed of a piezoelectric material such as a piezoelectric element, and can be driven at 0.1 nm/s or more. Examples of piezoelectric materials to be used include barium titanate (BaTiO₃), lead zirconate titanate (PZT), and zinc oxide (ZnO).

The terminal end of the DNA chain 116 and the surface of the biomolecule immobilizing member 202 are bonded each other via a covalent bond, an ionic bond, electrostatic interaction, magnetic force, and the like. For example, when the DNA chain 116 is immobilized by a covalent bond, the DNA chain 116 whose DNA terminal end is modified via APTES or glutaraldehyde is used. On the other hand, Si or SiO which acts as a scaffold for APTES is used on the surface of the biomolecule immobilizing member 202 for utilizing the above bond.

As another covalent bonding method, a gold thiol bond can be used. The 5′ end of the DNA chain 116 is modified with thiol, and gold is vapor-deposited on the surface of the biomolecule immobilizing member 202. Ag, Pt, and Ti to which thiol can be bonded can be utilize as another metal type that is vapor-deposited on the biomolecule immobilizing member 202.

A method for using an ionic bond is a method for immobilizing a negatively-charged biomolecule onto the surface of the biomolecule immobilizing member 202 that is positively charged by subjecting the biomolecule immobilizing member 202 to a surface modification treatment for positive charging in a solution. As a cationic polymer, polyaniline or polylysine is used.

In a method using electrostatic interaction, the DNA chain 116 whose amino terminal end is modified can be directly immobilized on an APTES-modified surface of the biomolecule immobilizing member 202. As a substrate surface, a nitrocellulose film, a polyvinylidene fluoride film, a nylon film, and a polystyrene substrate are widely used. In particular, a nitro cellulose film is used in the microarray technology. In use of a magnetic force, for example, the DNA chain 116 is previously immobilized on the surface of a magnetic bead by using the aforementioned bonding. A magnet material is further used as the biomolecule immobilizing member 202, whereby the magnetic bead having the DNA chain 116 immobilized thereon and the biomolecule immobilizing member 202 are interacted with each other and attraction of the DNA-immobilized magnetic bead by magnetic force is achieved. As a magnetic material, iron, silicon steel, amorphous magnetic alloy, a nanocrystal magnetic alloy, or the like is used.

Also in the case where a protein or an amino acid is measured as the DNA chain 116, the molecule may be modified at a specific bonding site and bonded to an immobilization substrate in the same manner. Thus, the bonding site in a protein can be identified and the sequence information of an amino acid can be acquired. The density of immobilization of the DNA chains 116 on the biomolecule immobilizing member 202 is determined depending on the spread of the electric field formed around the nanopore 113. A DNA solution containing the DNA chains 116 is injected into the liquid tank 112A through the opening provided for attachment of the biomolecule immobilizing member 202 and the driving mechanism 201.

In the above explanation, a structure where the biomolecule immobilizing member 202 and the driving mechanism 201 are attached to the device for biomolecule assays 110A is assumed, but these components do not have to be attached thereto in the distribution stage.

### (3-3) Array Passive Type

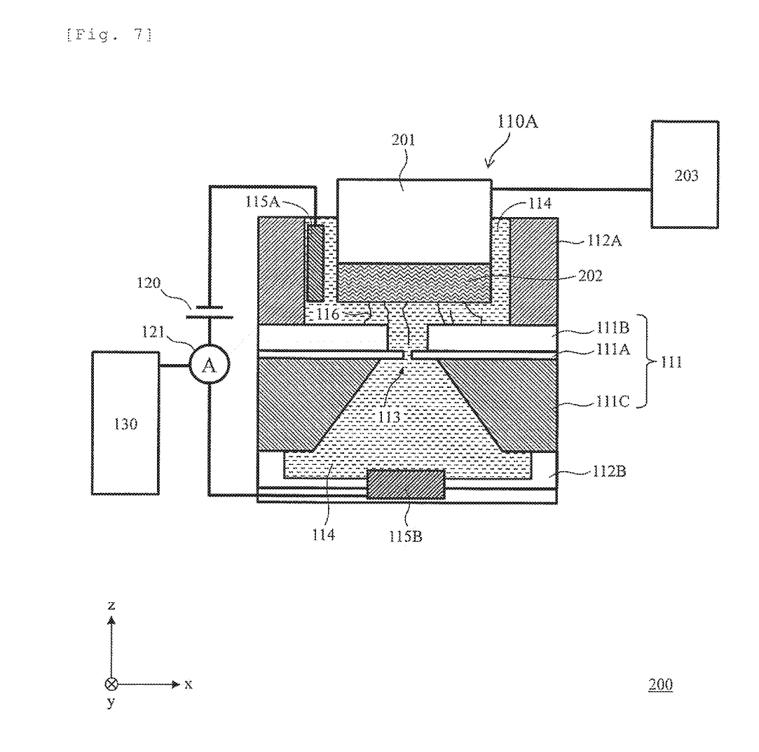

FIG. 8 shows a configuration example of a biomolecule assay apparatus 300 of an array passive type. In FIG. 8, the same sign is added to denote a component corresponding to a component in FIG. 6. An array type refers to an apparatus configuration where a plurality of the liquid tanks 112B are arranged for one liquid tank 112A. Such an array type is effective for simultaneously acquiring information on a larger number of the DNA chains 116.

In this embodiment, the membrane fixing member 111C includes four spaces separated by three partitions, each of the spaces is used as the liquid tank 112B. The liquid tank 112A is used as a common liquid tank for the four liquid tanks 112B positioned on the lower side.

In this embodiment, the single nanopore 113 and electrode 115B are provided in each of the liquid tanks 112B which are insulated from each other by the partitions. For this reason, a current flowing through each of the nanopores 113 can be independently measured. In this embodiment, the opening for attachment of the electrode 115A can be used as an inlet 301 for a DNA solution containing the DNA chains 116. That is, the DNA solution can be injected through the inlet 301.

### (3-4) Array Active Type

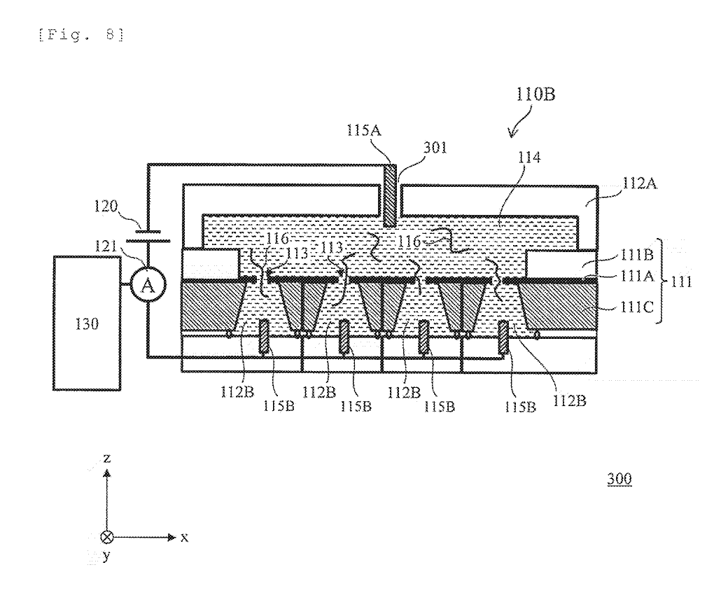

FIG. 9 shows a configuration example of a biomolecule assay apparatus 400 of an array active type. In FIG. 9, the same sign is added to denote a component corresponding to a component in FIG. 7. The arrow in FIG. 9 represents a change of the state when the biomolecule immobilizing member 202 is moved downwardly. In FIG. 9, the number of the biomolecule immobilizing member 202 is one, but a plurality of the biomolecule immobilizing members 202 maybe provided to correspond to the liquid tanks 112B. Of course, the DNA chains 116 are immobilized on a surface of the individual biomolecule immobilizing members 202 by the aforementioned technique. The plurality of the biomolecule immobilizing members 202 may be driven by one driving mechanism 201 or may be driven by the respective driving mechanisms 201.

### (4) Method of Fabrication of Device

Hereinunder, a method of fabrication of the devices for biomolecule assays 110 to 110C will be explained. A basic configuration itself of the devices for biomolecule assays 110 to 110C for use in assays of biopolymers by a so-called blockage current method is known in the art, and the components thereof can also be easily understood by those skilled in the art. For example, a specific device is disclosed in the U.S. Pat. No. 5,795,782, “Scientific Reports 4, 5000, 2014, Akahori, et al.”, “Nanotechnology 25(27): 275501, 2014, Yanagi, et al.”, “Scientific Reports, 5, 14656, 2015, Goto, et al.”, and “Scientific Reports 5, 16640, 2015”.

### Membrane

The membrane 111A in which the nanopore 113 is to be formed may be a lipid bilayer that has a pore at the center and comprises an amphiphilic molecular layer in which a protein is embedded (biopore) or a membrane of a material formed by a semiconductor microprocessing technique (solid pore). Examples of materials for membrane production by a semiconductor microprocessing technique include silicon nitride (SiN), silicon oxide (SiO₂), silicon oxide nitride (SiON), hafnium oxide (HfO₂), molybdenum disulfide (MoS₂), and graphene. The thickness of the membrane is angstrom to 200 nanometers, preferably 1 angstrom to 100 nanometers, more preferably 1 angstrom to 50 nanometers, and one example is about 5 nm.

For example, the membrane 111A is produced according to the following procedure. First, Si₃N₄/SiO₂/Si₃N₄ films are formed into 12 nm/250 nm/100 nm on a surface of a 8 inch Si wafer having a thickness of 725 μm, and an Si₃N₄ film is formed into 112 nm on the back surface. Next, a 500 nm square of the top Si₃N₄ film on the front surface is subjected to a reactive etching, and a 1038 nm square of the Si₃N₄ film on the back surface is subjected to a reactive ion etching. Furthermore, for the back surface, the Si substrate exposed by the etching is further etched with tetramethylammonium hydroxide (TMAH). During the Si etching, the wafer surface is covered with a protection film (ProTEKTMB 3 primer and ProTEKTMB 3, Brewer Science, Inc.) to prevent etching of SiO on the front surface side.

Next, the SiO layer exposed in an area of 500 nm square after removing the protection film was removed by a BHF solution (HF/NH₄F= 1/60, 8 min). This results in production of the partition 111 in which a membrane Si₃N₄ with a thickness of 12 nm was exposed. In this stage, no nanopore is provided in the membrane 111A.

### Nanopore Size

The size of the nanopore 113 can be appropriately selected according to the type of the biopolymer to be assayed, and is, for example, 0.9 nm to 100 nm, preferably 0.9 nm to 50 nm, and specifically approximately 0.9 nm or more and 10 nm or less or so. For example, the diameter of the nanopore 113 for use in an assay of ssDNA (single strand DNA) having a diameter of about 1.4 nm is preferably approximately 1.4 nm to 10 nm, more preferably approximately 1.4 nm to 2.5 nm, and specifically about 1.6 nm. For example, the diameter of the nanopore 113 for use in an assay of dsDNA (double strand DNA) having a diameter of about 2.6 nm is preferably approximately 3 nm to 10 nm, and more preferably approximately 3 nm to 5 nm.

The depth of the nanopore 113 can be adjusted by adjusting the thickness of the membrane 111A. The depth of the nanopore 113 is two times or more of a monomer unit constituting the biopolymer, preferably three times or more, and more preferably five times or more. For example, when the biopolymer is constituted of a nucleic acid, the depth of the nanopore 113 is preferably the length of 3 bases or more, for example, about 1 nm or more. Such a depth enables the biopolymer to enter the nanopore 113 with a controlled shape and migration velocity, and thus a high sensitive and high accurate analysis can be achieved. In addition, the shape of the nanopore 113 is basically circular, but elliptic or polygonal shapes may be adopted.

In the case of an apparatus configuration of an array type including a plurality of the membranes 111A each having the nanopore 113, the membranes 111A each having the nanopore 113 are preferably regularly arranged. The intervals between the plurality of the membranes 111A arranged may be 0.1 mm to 10 mm, and preferably 0.5 mm to 4 mm according to the ability of the electrodes and the electrical measurement system used.

### Formation of Nanopore

Examples of methods for forming the nanopore 113 in the membrane 111A include, but not limited to, electron beam irradiation by a transmission electron microscope or the like and insulation breakage by voltage application. For example, a method described in “Itaru Yanagi et al., Sci. Rep. 4, 5000 (2014)” can be used.

Formation of the nanopore 113 can be achieved, for example, by the following procedure. Before the partition 111 is set in the device for biomolecule assays 110 and the like, an Si₃N₄ membrane is hydrophilized using Ar/O₂ plasma (SAMCO Inc., Japan) under conditions of 10 WW, 20 sccm, 20 Pa, and 45 sec. Next, the partition 111 is set in the device for biomolecule assays 110. Then, the liquid tanks 112A and 112B are filled with a 1 M KCl, 1 mM Tris-10 mM EDTA solution of pH 7.5, the electrodes 115A and 115B are respectively introduced into the liquid tanks 112A and 112B.

A voltage is applied not only in formation of the nanopore 113 but also in measurement of the ion current flowing via the nanopore 113 after the formation of the nanopore 113. Here, the liquid tank 112B positioned on the lower side is called a cis tank and the liquid tank 112A positioned on the upper side is called a trans tank. A voltage Vcis applied to the electrode on the cis tank side is set to 0 V, and a voltage Vtrans is applied to the electrode on the trans tank side. The voltage Vtrans is generated from a pulse generator (41501B SMU AND Pulse Generator Expander, Agilent Technologies, Inc.).

After the pulse application, the current value was read by the ammeter 121 (4156B PRECISION SEMICONDUCTOR ANALYZER, Agilent Technologies, Inc.). A process of applying the voltage for formation of the nanopore 113 and a process of reading the ion current value are controlled by our company's own program (Excel VBA, Visual Basic for Applications). A current value condition (threshold current) is selected according to the diameter of the nanopore 113 formed before the application of the pulse voltage, and the diameter of the nanopore 113 is gradually increased to obtain an intended diameter.

The diameter of the nanopore 113 was estimated based on the ion current value. The standard for selecting the conditions is shown in Table 1.

TABLE 1
Conditions in voltage application

| Nonopore diameter before | Non-opening | | |
|---|---|---|---|
| application of pulse voltage | to 0.7 nmΦ | to 1.4 nmΦ | to 1.5 nmΦ |
| Applied voltage (V_{cis}) [V] | 10 | 5 | 3 |
| Initial application time [s] | 0.001 | 0.01 | 0.001 |
| Threshold current | 0.1 nA/0.4 V | 0.6 nA/0.1 V | 0.75 nA/0.1 V |

Here, the nth pulse voltage application time tₙ (n is an integer larger than 2) is determined by the following equation.

tₙ=10^{-3+(t/6×n-1)}−10^{-3+(1/6×n-2)} For n>2

The formation of the nanopore 113 can be achieved not only by the method of applying a pulse voltage but also by an electron beam irradiation by TEM (A. J. Storm et al., Nat. Mat. 2 (2003)).

When a voltage is applied to the electrodes 115A and 115B provided in the upper and lower two liquid tanks 112A and 112B from the power source 120, an electric field is generated in the vicinity of the nanopore 113 and the DNA chain 116 negatively charged in the solution passes through the nanopore 113. At this time, the blockage current I_{b} described above (FIG. 1-1) flows.

### Liquid Tank

The liquid tanks 112A and 112B which can store a measurement solution in contact with the membrane 111A can be appropriately provided by such a material, shape, and size that have no influence on measurement of a blockage current. A measurement solution is injected so as to come into contact with the membrane 111A separating the liquid tanks 112A and 112B.

### Electrode

Electrodes 115A and 115B are preferably made of a material that can undergo an electron transfer reaction (Faraday reaction) with electrolytes in a measurement solution, and are typically made of silver halide or alkali halide/silver. From the viewpoint of potential stability and reliability, silver or silver/silver chloride is preferably used.

The electrodes 115A and 115B may be made of materials that are to be polarized electrodes, and may be, for example, gold, platinum, and the like. In this case, a material that can assist the electron transfer reaction, for example, potassium ferricyanide or potassium ferrocyanide is preferably added to the measurement solution for securing a stable ion current. Alternatively, a material that can undergo an electron transfer reaction, for example, a ferrocene is preferably fixed on a surface of a polarized electrode.

The entire structures of the electrodes 115A and 115B may be made of the material, or the material may be applied on the surface of a base material (copper, aluminum, etc.). The shape of the electrodes is preferably, but not limited to, a shape providing a large surface area to be in contact with the measurement solution. The electrodes are bonded to wires and electrical signals are sent to a measurement circuit.

### (5) Use Example of Electrolyte Solution

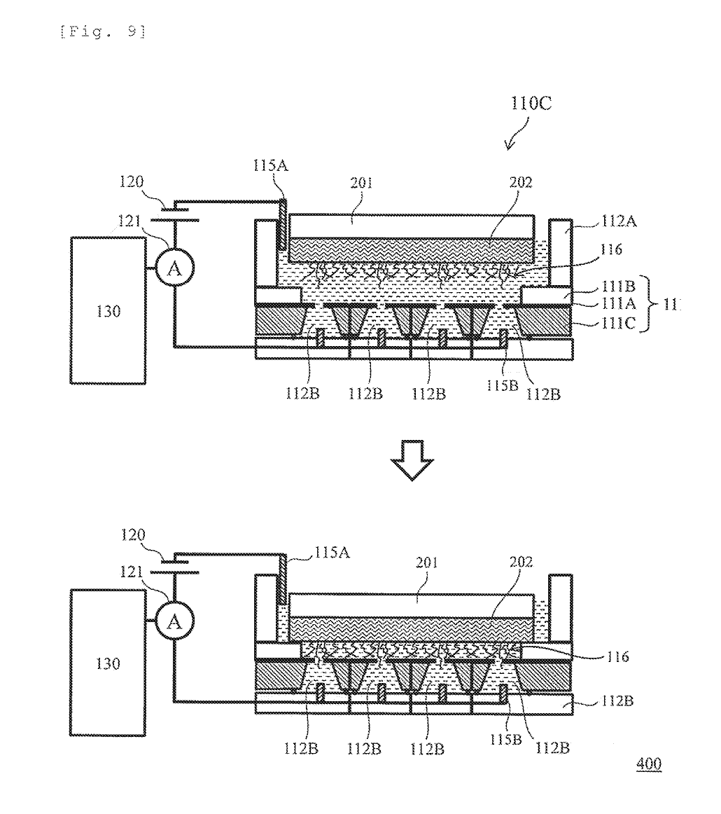

Finally, examples of the use of the electrolyte solution 114 having the composition according to the example described in “(2) Electrolyte Solution” will be confirmed. FIG. 10 shows a procedure in the case without replacement of the solution after formation of the nanopore 113. In this case, first, the liquid tanks 112A and 112B are filled with the electrolyte solution 114 having the composition according to the example described in “(2) Electrolyte Solution” (1001). Next, a voltage is applied to the electrodes 115A and 115B to form the nanopore 113 (1002). In the stage where the nanopore 113 having a suitable diameter is obtained, a feature analysis processing of a biomolecule is performed (1003).

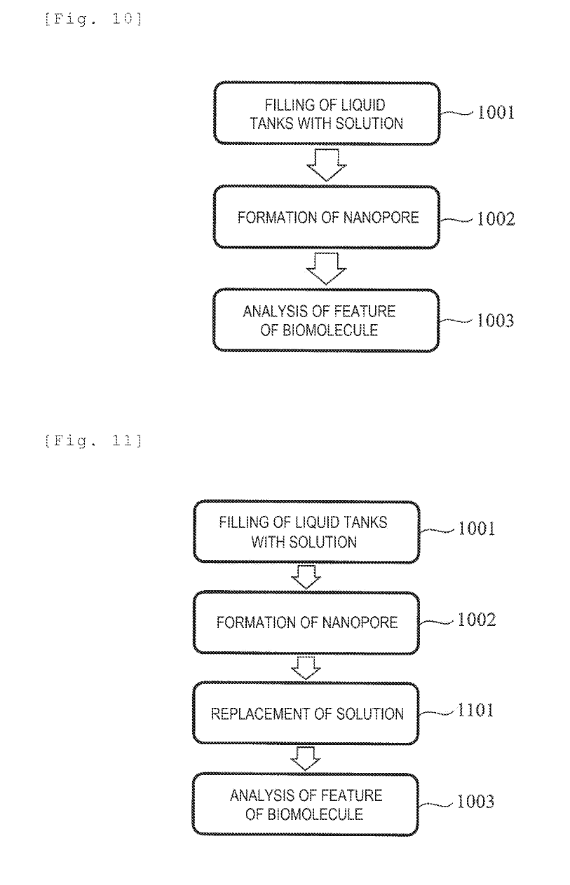

FIG. 11 shows a procedure in the case with replacement of the solution after formation of the nanopore 113. In FIG. 11, the same sign is added to denote a step corresponding to a step in FIG. 10. In this case, when the nanopore 113 is formed in the processing 1002, the electrolyte solution 114 in the liquid tanks 112A and 112B is replaced (1101). The electrolyte solution 114 that is newly introduced may be the electrolyte solution 114 having the composition according to the example described in “(2) Electrolyte Solution” or maybe an existing electrolyte solution, as described above.

### (6) Other Examples

The present invention is not limited to the foregoing embodiments and includes various modification examples. For example, the foregoing embodiments are described in detail for explaining the present invention in an easy-to-understand manner, and the present invention is not necessarily include all the configurations described above. A part of an embodiment may be replaced with a part of another embodiment. A configuration of an embodiment may be added to a configuration of another embodiment. A part of a configuration of each embodiment may be added to, deleted from, or replaced with a part of a configuration of another embodiment.

### REFERENCE SIGNS LIST

100, 200, 300, 400 Biomolecule assay apparatus,

111 Partition,

111A Membrane,

111B, 111C Membrane fixing member,

112A, 112B Liquid tank,

113 Nanopore,

114 Electrolyte solution,

115A, 115B Electrode,

116 DNA Chain,

120 Power source,

121 Ammeter,

130 Computer,

201 Driving mechanism,

202 Biomolecule immobilizing member,

203 Control unit,

301 Inlet

1. An electrolyte solution for biomolecule assays, comprising
D₂O as a solvent, and/or
an electrolyte that has Cs and Na, Na alone, Na and Li, or Li alone as a cation species in the electrolyte solution, or trishydroxyaminomethane, or a combination thereof.

2. The electrolyte solution for biomolecule assays according to claim 1, comprising a halide ion as an anion of the electrolyte.

3. The electrolyte solution for biomolecule assays according to claim 1, wherein a pH of the electrolyte solution is a pKa of a guanine base or higher and pH 14 or lower.

4. The electrolyte solution for biomolecule assays according to claim 1, wherein a pH of the electrolyte solution is a pKa of a guanine base or higher and pH 12 or lower.

5. The electrolyte solution for biomolecule assays according to claim 1, wherein a concentration of a pH modifier for the electrolyte solution is 100 mM or more.

6. The electrolyte solution for biomolecule assays according to claim 1, wherein a total concentration of the cation species is 0.1 M or higher and the saturation concentration or lower.

7. A device for biomolecule assays, comprising
a first liquid tank filled with an electrolyte solution,
one or a plurality of second liquid tanks filled with an electrolyte solution,
a membrane that separates the electrolyte solution filled in the first liquid tank and the electrolyte solution filled in the one or plurality of second liquid tanks,
a first electrode provided in the first liquid tank, and
a second electrode provided in the second liquid tank, the electrolyte solution comprising
D₂O as a solvent, and/or
an electrolyte that has Cs and Na, Na alone, Na and Li, or Li alone as a cation species in the electrolyte solution, or trishydroxyaminomethane, or a combination thereof.

8. The device for biomolecule assays according to claim 7, wherein the membrane has a nanopore formed therein.

9. The device for biomolecule assays according to claim 7, wherein the first liquid tank has an opening for introducing into the tank a biomolecule immobilizing member for immobilizing a biomolecule to be assayed thereon.

10. A biomolecule assay apparatus, comprising
a device for biomolecule assays, including a first liquid tank filled with an electrolyte solution, one or a plurality of second liquid tanks filled with an electrolyte solution, a membrane that separates the electrolyte solution filled in the first liquid tank and the electrolyte solution filled in the one or plurality of second liquid tanks, and a first electrode provided in the first liquid tank and a second electrode provided in the second liquid tank; and
a measuring unit for measuring an ion current flowing between the first electrode and the second electrode, the electrolyte solution comprising
D₂O as a solvent, and/or
an electrolyte that has Cs and Na, Na alone, Na and Li, or Li alone as a cation species in the electrolyte solution, or trishydroxyaminomethane, or a combination thereof.

11. The biomolecule assay apparatus according to claim 10, wherein
the first liquid tank has
an opening for introducing into the tank a biomolecule immobilizing member for immobilizing a biomolecule to be assayed thereon, and further comprises
a driving mechanism for driving the biomolecule immobilizing member closer to or further from the membrane, and
a control unit for controlling an operation of the driving mechanism.

12. The biomolecule assay apparatus according to claim 11, wherein a surface of the biomolecule immobilizing member that faces the membrane has an area larger than a through hole formed in a membrane fixing member for fixing the membrane.

13. The biomolecule assay apparatus according to claim 10, wherein a biomolecule to be assayed is a nucleic acid or a protein.

14. The biomolecule assay apparatus according to claim 10, wherein the measuring unit controls a processing for forming a nanopore under a condition where the electrolyte solution is in contact with the membrane before the measurement of the ion current is started.

15. The biomolecule assay apparatus according to claim 10, wherein the measuring unit assays a biomolecule to be assayed based on a variation in the ion current measured.
